(19) United States
(12) Patent Application Publication
(10) Pub. No.: US 2018/0042515 A1
(43) Pub. Date: Feb. 15, 2018

(54) TRACKING SIGNALS FOR CATHETER

(71) Applicant: IMRICOR MEDICAL SYSTEMS, INC., BURNSVILLE, MN (US)

(72) Inventors: Steven R. Wedan, Savage, MN (US); Thomas W. Lloyd, Eagan, MN (US); Milton Noe Turcios, Farmington, MN (US); Daniel N. Sunnarborg, Bloomington, MN (US)

(21) Appl. No.: 15/556,890

(22) PCT Filed: Mar. 9, 2016
(86) PCT No.: PCT/US2016/021467

§ 371 Date: Sep. 8, 2017

Related U.S. Application Data
(63) Continuation-in-part of application No. 14/642,049, filed on Mar. 9, 2015, now abandoned.

(51) Int. Cl. A61B 5/06; G01R 33/28; A61B 34/20

(52) U.S. Cl. CPC A61B 5/062; A61B 34/20; G01R 33/287; A61B 2034/2051

(57) ABSTRACT

A method for projecting a broad tracking signal received by an inductively coupled element, such as a transformer during an MR tracking sequence is provided. By varying projection planes, the signal acquired from the transformer along the transmission line can be used to depict the body of the actively tracked medical device, such as the shaft or deflection region of a catheter. This may be achieved by interpolating a line between the position of the transformer element within the transmission line and the tracking coil. A curvature may be added to the line segment and gradually increased until the arc length of the line segment is approximately equal to the predefined length. The direction of the curve may be determined by virtually connecting the transformer position to the distal most tracking coil position, then the curve of the line segment is increased towards the proximal coil position.

### FIELD OF THE INVENTION

The present invention relates generally to a method for projecting a broad tracking signal received by an inductively coupled element, such as a transformer during an MR tracking sequence.

### BACKGROUND OF THE INVENTION

Interventional medical procedures are typically performed using x-ray fluoroscopy imaging to guide the procedure. X-ray imaging is used to visualize devices and anatomy inside a patient. However, because of its superior soft tissue imaging capabilities, many procedures can benefit from the utilization of magnetic resonance imaging (MRI) for guidance rather than x-ray imaging.

In many MRI guided interventional procedures, it is advantageous to locate the device within the patient using active MR tracking. Active MR tracking is a well-known technique wherein one or more MR receive coils (“tracking coils”) are incorporated into a medical device, and tracking pulse sequences or projections generated by the MRI machine are used to locate the coils. Tracking pulse sequences generally locate a tracking coil by finding the location of the tracking coil in each of three orthogonal planes. Such planes correspond to an x-y-z coordinate system, but the relationship between this x-y-z coordinate system and the patient or MR system may be arbitrary.

The MR tracking pulse sequences generate signals in the tracking coils, which are transmitted to electronic circuity connected to the tracking coils. This connection is often facilitated by a transmission line, such as a coaxial cable. However, if the length of the coaxial cable is sufficiently long, safety issues arise due to radiofrequency (RF) coupling to the transmission line. Several techniques have been proposed to make transmission lines safe for use in MRI. One technique incorporates miniature inductively coupled elements, such as transformers, along the transmission line to reduce the common mode RF currents.

However, an inductively coupling element (ICE), such as a transformer, may pick up MR signals from surrounding tissues, and therefore behave as tracking coils themselves. In other words, the ICE is acting as a tracking element and receiving MR signals in a manner similar to the tracking coils. Depending on the orientation of the ICE relative to the tracking sequence pulses or projections, the MR signal received from the ICE may have equal or greater signal intensity than the MR tracking signal picked up from the MR tracking coil. If the signal from the ICE is larger or has greater signal intensity than the signal received by the MR tracking coil, the location of the tracking coil may be erroneously determined to be location of the ICE. This, in turn, results in an erroneous tracking location for the medical device in which the tracking coil is embedded. In addition, even in the absence of any ICE, the signal received by MR tracking coils may have a variable magnitude and/or distribution, depending on its orientation to the coordinate system used to generate projections from a tracking sequence.

Thus, what is needed is an improved method and system for generating tracking projections along multiple projection axes to identify broad or varying tracking signals received by tracking elements such as tracking coils, transformers, or other inductively coupled elements, during an MR tracking sequence.

### BRIEF SUMMARY OF THE INVENTION

The foregoing problems with conventional systems are addressed with the system and method in accordance with the invention.

The method for projecting a broad tracking signal received by an inductively coupled element, such as a transformer during an MR tracking sequence and using the projection to depict the body of the device containing said inductively coupled device in accordance with the invention includes varying one or more projection planes of a tracking signal; acquiring a tracking signal from a tracking element along a transmission line to depict a body of an actively tracked medical device; interpolating a line between a position of the transformer element within the transmission line and a tracking coil; adding a curvature to a line segment and increasing the curvature until an arc length of the line segment is approximately equal to a predefined length; determining a direction of the curvature by virtually connecting a position of the transformer to a distal most tracking coil position; and increasing the curvature of the line segment towards a proximal tracking coil or other tracking element within the device.

The accuracy of the depiction of the device body can be improved by determining the precise 3-dimensional location of the individual tracking elements within the device such as transformers and tracking coils by varying the one or more projection planes a sufficient number of times to generate the projections that characterize the device. In the case of transformers comprising elongated loops, the characteristic projections would be narrow band, high amplitude spikes in two planes, and broad lower amplitude plane in a third plane as shown in FIG. 2 and FIG. 3.

The method in accordance with the invention may be used to identify individual components within a device and depict the body of a device between individual components.

The system in accordance with the invention includes tracking elements capable of receiving signals from an MRI, a device housing such components, an MRI for generating tracking signals, and computer capably of processing the signals received from the components and generating tracking projections.

While methods for projecting a broad signal associated with an inductively coupled element have been presented, those of skill in the art will appreciate that similar methods may be used to project signals with varying distributions and intensity levels generated by a tracking elements, such as tracking coils and other structures that act as antennas in an MRI.

Other aspects of the invention can be found in the following numbered clauses:

1. A method for determining the position of a tracking coil or inductively coupled element, such as a transformer during an MR tracking sequence comprising:

acquiring a first tracking signal from the tracking coil or inductively coupled element;

varying one or more of the projection planes of the tracking sequence to create a rotated tracking coordinate system;

acquiring a second tracking signal using the rotated tracking coordinate system from the tracking coil or inductively coupled element;

interpreting the two tracking signals to determine the location of the tracking coil or inductively coupled element;

using the location of the tracking coil or inductively coupled element to track or guide a medical device during a medical procedure.

2. The method of clause 1, wherein one or more of the projection planes are varied one or more times and two or more tracking signals are acquired.
3. The method of clause 2, wherein one or more projection planes are used to acquire tracking signals from two or more tracking elements such as a tracking coils or inductively coupled elements.
4. The method of clause 3, wherein the locations of the elements are used to depict the body of the medical device by:

interpolating a line between a position of a first tracking element associated with the device and a second tracking element associated with the device;

adding a curvature to the line segment and increasing the curvature until an arc length of the line segment is approximately equal to the predefined device length separating the two tracking elements;

determining the direction of curvature by intersecting the line segment with a third tracking element located between the first and second tracking elements.

5. The method of clause 2, wherein tracking signals are acquired along a sufficient number of projection planes to identify projections characteristic of the tracking element.
6. The method of clause 5, wherein the characteristic projections of a tracking element are used to determine the orientation of the element in addition to its location.
7. The method of clause 5, wherein the characteristic projections of a tracking element are used to identify specific features of the tracking element.
8. The method of clause 5, wherein the characteristic projections of a tracking element are used to identify specific regions of the tracking element.
9. The method of clause 3, wherein the locations of the elements are used to depict the body of the medical device by:

interpolating a line between a position of a first tracking element associated with the device and a second tracking element associated with the device;

adding a curvature to the line segment and increasing the curvature until an arc length of the line segment is approximately equal to the predefined device length separating the two tracking elements;

using the characteristic projections of one or more of the tracking elements to determine the orientation of the tracking element in 3D space;

using the orientation of one or more of the tracking projections to determine the trajectory of the line segment near the one or more tracking elements;

using the trajectory of the line segment near one or more tracking elements to determine the curvature of the line segment between the first and second tracking elements.

10. The method of clause 3, wherein the multiple tracking signals are used to determine the location of a tracking element that produces broad projections in certain projection planes and distinct projections in other projection planes that would result in intermittent tracking depending on the orientation of the tracking element.
11. The method of clause 3, wherein the multiple tracking signals are used to determine the orientation of a tracking element that produces both broad and distinct projections and the multiple tracking signals are used to determine the orientation of the object by using the two sharp projections to determine the coordinates of the object in two planes and the broad projection is used to determine the range of coordinates in the third plane.
12. The method of clause 2, wherein the multiple tracking signals are used to improve the accuracy of the tracking element location.
13. The method of clause 2, wherein the multiple tracking signals are used to reduce loss of tracking element location associated with tracking element orientations that minimize projections along one or more projection planes.

### BRIEF DESCRIPTION OF THE DRAWINGS

For a better understanding of the invention, and to show how the same may be carried into effect, reference will now be made, by way of example, to the accompanying drawings, in which:

FIG. 1 depicts an example of an x-y-z coordinate system and an alternative orthogonal x′-y′-z′ coordinate system in dashed lines that is rotationally offset from the original x-y-z coordinate system.

FIG. 2A-2C illustrates three orientations for an tracking element comprising an ICE, or a transformer, with elongated loops relative to the MR gradient along the projection axis used for tracking and shows ICE orientations and the characteristic projections that maximize and minimize signals pickup by the ICE.

FIG. 3A-3C further illustrates a transformer or ICE, which can act as a tracking element and illustrates the tracking coordinate system that results in characteristic projections of the tracking element. Orientating the tracking coordinate system such that the tracking signal consists of characteristic projections of the tracking element, allows for the identification of the tracking element and the determination of its position and orientation in 3D space.

FIGS. 4A-4B depict a conventional system with an ICE and tracking coil projecting a signal onto the z-axis and showing that the z-axis projection of the ICE is comparable in amplitude to the projection from the tracking coil signals along the same axis.

FIGS. 5A-5B depict the orientations of the ICE and tracking coil relative to the tracking projection showing that the tracking coil signal has a substantially larger magnitude and is easily distinguishable from the ICE signal, in accordance with the invention.

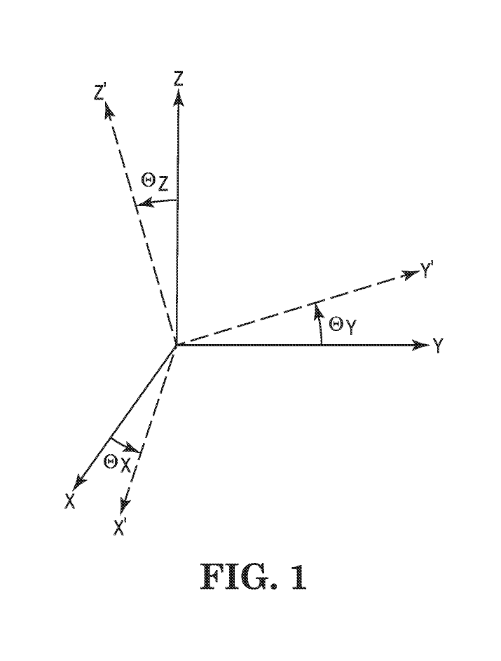

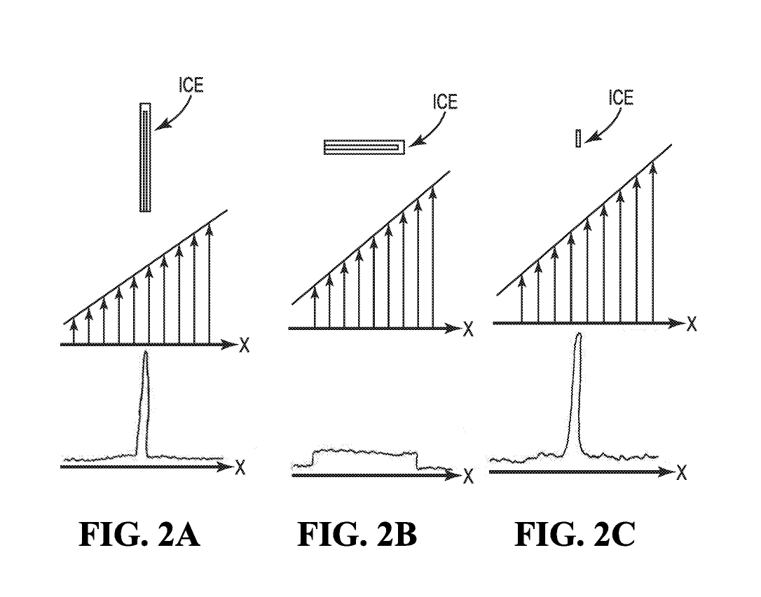

### DETAILED DESCRIPTION OF THE INVENTION

The MR signal intensity received by an inductively coupled element (ICE), such as a transformer, during an MR tracking sequence is dependent on the relative orientation of the ICE and the tracking pulse sequence projection planes. For instance, when an MR tracking plane projection is oriented orthogonally to the long axis of an ICE, then the magnetic field present at tissues surrounding the ICE structure may be substantially the same, resulting in a large projection signal from the ICE in that plane. Alternately, if the MR tracking plane projection is parallel to the long axis of the ICE, then the tissues around the ICE will have different magnetic fields applied to them by the MR gradient system, resulting in a lower and more broad signal projection received by the ICE.

Similarly, an MR tracking coil may receive varying levels of MR signal from surrounding tissues depending on its physical characteristics and relative orientation to the tracking coil projections. In some orientations, the MR tracking signal received by the tracking coil may have an irregular distribution along the projection axis or have a magnitude too low to detect.

Therefore, in a device that utilizes an MR tracking coil with an ICE in the transmission line, the ICE location can be mistaken for the tracking coil locations, depending upon the orientation of the device, resulting in a false location of the device.

Similarly, in a device that utilizes an MR tracking coil without an ICE in the transmission line, tracking effectiveness can be variable depending upon the orientation of the device, resulting in loss of tracking.

Because device orientation is often variable throughout a procedure, a single set of tracking projection planes is not always optimal.

The present invention is a system that uses varying projection planes to track medical devices. In one embodiment, each tracking pulse sequence uses one or more different projection plane(s). In this way, the probability of prolonged false or poor device tracking is minimized, since the relative orientation of the device to the tracking projection plane(s) is continuously variable.

In another aspect of the invention multiple projection planes are used to identify and localize specific components in a medical device based on characteristics related to the tracking projection of the component.

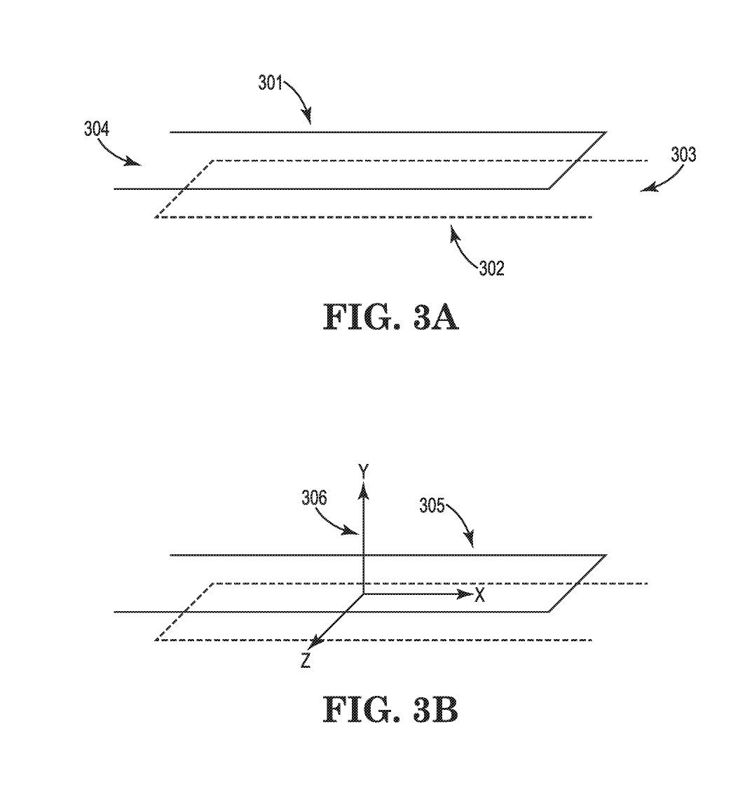

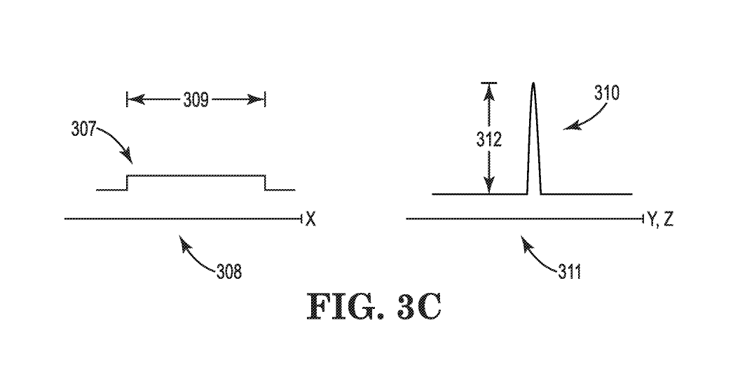

The accuracy of the depiction of the tracking element and the body of the device can be increased by acquiring projections along multiple projection planes such that the characteristic projections of elements within the device are identified. These characteristic projections can be used to determine the element orientation in addition to the element location. Doing so limits the potential trajectories of the line used to connect individual elements to those that physically agree with the individual element orientations. This is further illustrated in FIGS. 3A-3C.

Referring now to FIGS. 3A-3C, an inductively coupled element that can act as a tracking element and has characteristic projections allowing the orientation of the element to be determined by processing multiple tracking signals with varying projection planes.

FIG. 3A illustrates an inductively coupled device that acts as a tracking element and a transformer that can be placed in the transmission line connecting a tracking coil to external receive circuitry. A loop 301 forms the first side of the transformer. A second loop 302 forms the second side of the transformer. When placed in a transmission line, the transformer is connected to the transmission line at ends 303 and 304.

FIG. 3B illustrates the orthogonal tracking coordinate system 306 comprising the three projection axes (x, y, and z) defining the MRI gradient field orientations that result in the three characteristic projections associated with the tracking element 305.

FIG. 3C illustrates the characteristic projections from tracking element 305 in FIG. 3B. Tracking projection 307 along tracking axis 308 has a characteristic width 309 when the tracking coordinate system is oriented such that the x axis of 306 is parallel with the long axis of the tracking element 305. In this configuration, the width of the tracking projection is maximized.

FIG. 3C also illustrates the characteristic projection 310 associated with the y axis and z axis 311 when the tracking coordinate system 306 is oriented as shown in FIG. 3B. In this configuration, the characteristic projection along the y axis and z axis 311 consists of a sharp spike 310 with the amplitude 312 maximized and the bandwidth minimized.

In the following examples, only one projection plane is used, such that the problem of conventional systems and the solution to the problem in accordance with the invention can be described more easily with reference to the figures.

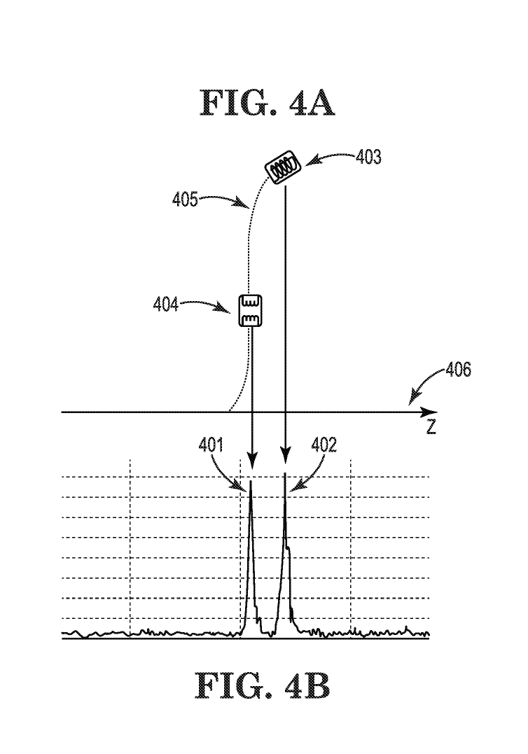

Referring now to FIGS. 4A and 4B, the orientation of the z-axis along which the z-projection is acquired is shown at 406. Transmission line 405 with integrated ICE 404 connects the tracking coil 403 to receive circuitry. ICE signal 401 is projected on the z-axis 406. Tracking coil signal 402 received by tracking coil 403 is also projected on the z-axis 406. The signal 401 received by the ICE 404 along the z-axis is comparable in magnitude 401 to the tracking coil signal 402 received by the tracking coil 403. This is a result of the ICE being oriented sympathetically to the magnetic field gradient, whereas the tracking coil is not. In such a configuration the ICE 404 may be misidentified as the tracking coil 403 resulting in tracking errors.

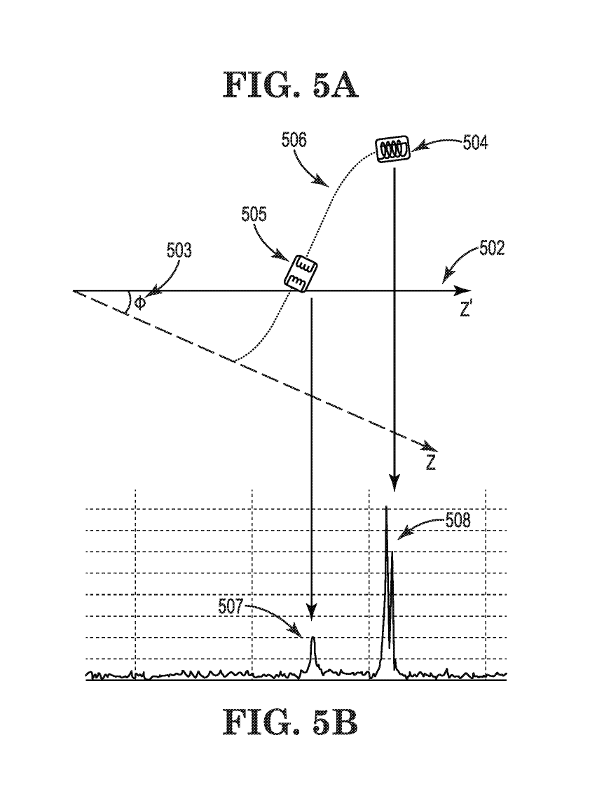

Referring now to FIGS. 5A and 5B, the system and method in accordance with the invention is depicted. The orientation of the z-axis along which the z-projection is acquired is depicted at 501 (equivalent to 406 in FIG. 4B). The orientation of the z′-axis after a shift of 4) degrees in the xy plane is shown at 502. The angle φ between the z-axis along which the z projection is acquired and the z′-axis along which the z′-projection is acquired is shown at 503. Transmission line 506 with integrated ICE 505 connects the tracking coil 504 to receive circuity. Signal 507 received by the ICE along the z′ projection 502 is shown. The signal received by the tracking coil 508 along the z′ projection 502 is also shown. As can be seen, the ICE signal 507 is lower in magnitude than the tracking coil signal 508, which is greater in magnitude. The orientations of the ICE and tracking coil, relative to the tracking projection, are such that the tracking coil signal has a magnitude greater than the ICE signal, which results in the tracking coil being correctly tracked.

The foregoing increase in magnitude of the tracking coil signal over the ICE signal is accomplished by varying one or more projection planes of a tracking signal. Further, by acquiring a tracking signal from a transformer or other element along a transmission line it is possible to depict a body of an actively tracked medical device by interpolating a line between a position of the transformer or element within the transmission line and a tracking coil; adding a curvature to a line segment and increasing the curvature until an arc length of the line segment is approximately equal to a predefined length; determining a direction of the curvature by virtually connecting a position of the transformer to a distal most tracking coil position; and increasing the curvature of the line segment towards a proximal coil position.

As noted the foregoing examples used one plane. However, by creating and continually using varying projection planes, the system in accordance with the invention can either collect several sets of projections before determining the location of the tracking coil and inductively coupled elements, or it can determine the location of the elements with each projection and check for inconsistencies between projections before rendering the final tracking coil location.

A further advantage of using varying projection planes is that the signal acquired from the tracking elements along the transmission line can be used to depict the body of the actively tracked medical device, such as the shaft or deflection region of a catheter. This can be achieved by interpolating a line between the position of the transformer element within the transmission line and the tracking coil. A curvature can be added to the line segment and gradually increased until the arc length of the line segment is approximately equal to the predefined length. The direction of the curve can be determined by virtually connecting the transformer position to the distal most tracking coil position, then the curve of the line segment is increased towards the proximal coil position. The accuracy of the depiction can be improved by determining the orientation of the inductively coupled elements by using multiple projection planes to identify characteristic projections from the elements.

Using multiple different projection planes (or orthogonal coordinate systems) for the acquisition of tracking signal data also allows for the following:

The ability to utilize averaging of multiple calculated tracking element positions to minimize errors associated with any single acquisition/calculation.

The ability to utilize multiple projections from a single tracking coil or inductively coupled element with an understanding of the sensitivity of the tracking element to identify specific aspects, or characteristic projections, of the element, such as the location of its edges, center, or orientation.

The ability to allow a single tracking element to provide multiple tracking locations. By way of example, the edge and center of the coil could be used as a distinct tracking locations.

Multiple projections could also be used with signal processing to allow individual tracking coils to be located closer to one another.

It is contemplated that communicating with multiple tracking coils using a single transmission line and imaging using tracking coils for receive antennas to produce a region of high intensity in the MR image and using that high intensity region to identify the catheter and/or tracking coils fall within the scope of the invention.

The invention also includes a computer having memory and a processor operably coupled to software that runs one or more of the following functions: generate and/or optimize varying projection planes and tracking coordinate systems; identify predefined characteristic projections; optimize tracking accuracy based on the projections; determine the device orientation based on the characteristic projections; and create a depiction of the medical device.

Although the present invention has been described with reference to particular embodiments, those of ordinary skill in the art will recognize that changes may be made in form and detail without departing from the spirit and scope of the invention.

What is claimed is:

1. A method for determining the position of a tracking coil or inductively coupled element, such as a transformer during an MR tracking sequence comprising:
acquiring a first tracking signal from the tracking coil or inductively coupled element;
varying one or more of the projection planes of the tracking sequence to create a rotated tracking coordinate system;
acquiring a second tracking signal using the rotated tracking coordinate system from the tracking coil or inductively coupled element;
interpreting the two tracking signals to determine the location of the tracking coil or inductively coupled element;
using the location of the tracking coil or inductively coupled element to track or guide a medical device during a medical procedure.

2. The method of claim 1, wherein one or more of the projection planes are varied one or more times and two or more tracking signals are acquired.

3. The method of claim 2, wherein one or more projection planes are used to acquire tracking signals from two or more tracking elements such as a tracking coils or inductively coupled elements.

4. The method of claim 3, wherein the locations of the elements are used to depict the body of the medical device by:
interpolating a line between a position of a first tracking element associated with the device and a second tracking element associated with the device;
adding a curvature to the line segment and increasing the curvature until an arc length of the line segment is approximately equal to the predefined device length separating the two tracking elements;
determining the direction of curvature by intersecting the line segment with a third tracking element located between the first and second tracking elements.

5. The method of claim 2, wherein tracking signals are acquired along a sufficient number of projection planes to identify projections characteristic of the tracking element.

6. The method of claim 5, wherein the characteristic projections of a tracking element are used to determine the orientation of the element in addition to its location.

7. The method of claim 5, wherein the characteristic projections of a tracking element are used to identify specific features of the tracking element.

8. The method of claim 5, wherein the characteristic projections of a tracking element are used to identify specific regions of the tracking element.

9. The method of claim 3, wherein the locations of the elements are used to depict the body of the medical device by:
interpolating a line between a position of a first tracking element associated with the device and a second tracking element associated with the device;
adding a curvature to the line segment and increasing the curvature until an arc length of the line segment is approximately equal to the predefined device length separating the two tracking elements;
using the characteristic projections of one or more of the tracking elements to determine the orientation of the tracking element in 3D space;
using the orientation of one or more of the tracking projections to determine the trajectory of the line segment near the one or more tracking elements;
using the trajectory of the line segment near one or more tracking elements to determine the curvature of the line segment between the first and second tracking elements.

10. The method of claim 3, wherein the multiple tracking signals are used to determine the location of a tracking element that produces broad projections in certain projection planes and distinct projections in other projection planes that would result in intermittent tracking depending on the orientation of the tracking element.

11. The method of claim 3, wherein the multiple tracking signals are used to determine the orientation of a tracking element that produces both broad and distinct projections and the multiple tracking signals are used to determine the orientation of the object by using the two sharp projections to determine the coordinates of the object in two planes and the broad projection is used to determine the range of coordinates in the third plane.

12. The method of claim 2, wherein the multiple tracking signals are used to improve the accuracy of the tracking element location.

13. The method of claim 2, wherein the multiple tracking signals are used to reduce loss of tracking element location associated with tracking element orientations that minimize projections along one or more projection planes.
